SEQUENCE LISTING (15 sequences)

SEQ ID NO: 1 - protein, 416 aa, S. cerevisiae
MIDRTELSKFGITTQLSVIGRNPDEQSGFVNPPLYKGSTIILKKLSDLEQRKGRFYGTAGSPTIDNLENAWTHLTGGAGTVLSASGLGSISLALLALSKAGDHILMTDSVYVPTRMLCDGLLAKFGVETDYYDPSIGKDIEKLVKPNTTVIFLESPGSGTMEVQDIPALVSVAKKHGIKTILDNTWATPLFFDAHAHGIDISVEAGTKYLGGHSDLLIGLASANEECWPLLRSTYDAMAMLPGAEDCQLALRGMRTLHLRLKEVERKALDLAAWLGNRDEVEKVLHPAFEDCPGHEYWVRDYKGSSGLFSIVLKNGFTRAGLEKMVEGMKVLQLGFSWGGYDSLITPLNPCNNRKASTWPYKGFALRIQVGLEEFEDLKRDFELGFERLAEEISLNPLQIFFFLFFLDFQAMLLRN

SEQ ID NO: 2 - DNA, 1253 bp, S. cerevisiae
atgattgatcgtaccgagttatcgaagtttggtattactacgcaactgtctgttattggacgtaatccagatgaacaaagtggctttgttaatccacctttgtataaggggtcaaccatcattcttaaaaaacttagtgatttagaacaaaggaaaggaagattttacgggacagcaggttctccaactattgacaatttagaaaatgcctggacgcatttaaccggcggcgctgggacagtgctatcagcttctgggcttggttctatctctttggcgctattggccctttcgaaagctggtgatcatatcttgatgactgatagtgtctacgtgccaacacgtatgctatgtgatggtttattggccaagttcggtgttgaaacggattattatgacccatcaatagggaaggatatagaaaaactagttaagccaaatacaaccgtcattttcctcgaaagcccgggttctgggaccatggaagtacaggatattccagctttggtctctgttgccaaaaagcatgggataaagacaattctagacaacacatgggcaacgccactcttttttgatgctcatgcgcatggtatcgatatttcggtagaagctgggacaaaatatttgggtggtcattcagatcttcttattggtctggcctccgcaaatgaagaatgttggccgctattacggtcaacttatgatgcaatggcaatgttaccaggtgccgaggactgtcaattagcattgcgaggaatgcgtacattgcacttaagattgaaagaggtagaaagaaaagccctggatttggctgcttggctcggaaatcgagatgaggttgaaaaagtgcttcaccccgcctttgaagattgtcccggacatgaatactgggttcgtgactacaaaggttcttcaggcttattttccattgtccttaaaaatgggttcacaagagctggtctggagaaaatggtagaagggatgaaagttttgcaattgggattttcatggggtggctacgactccttgattacccctttaaatccttgtaataatagaaaagcttcaacatggccttacaaaggttttgcactaagaatacaagtgggtctcgaagaatttgaagatttaaaaagggattttgagttaggctttgaacgtctcgcagaagaaatttctttgaatcctttacaaatctgattttttttccttttttttttggatttccaagcaatgttgttgagaaatga

SEQ ID NO: 3 - protein, 416 aa, S. cerevisiae
MIDRTELSKFGITTQLSVIGRNPDEQSGFVNPPLYKGSTIILKKLSDLEQRKGRFYGTAGSPTIDNLENAWTHLTGSAGTVLSASGLGSISLALLALSKADDHILMTDSVYVPTRMLCDGLLAKFGVETDYYDPSIGKDIEKLVKPNTTVIFLESPGSGTMEVQDIPALVSVAKKHGIKTILDNAWATPLFFDAHAHGIDISVEAGTKYLGGHSDLLIGLASANEECWPLLRSTYDAMAMLPGAEDCQLALRGMRTLHLRLKEVERKALDLAAWLGNRDEVEKVLHPAFEDCPGHEYWVCDYKGFSGLFSIVLKNGFTRAGLEKMVEGMKVLQLGFSWGGYDSLITPVNPCNNRKASTWPYKGFALRIQVGLEEFEDLKRDFELGFERLAEEISLNPLQIFFFLFFLDFQAMLLRN

SEQ ID NO: 4 - DNA, 1253 bp, S. cerevisiae
atgattgatcgtaccgagttatcgaagtttggtattactacgcaactgtctgttattggacgtaatccagatgaacaaagtggctttgttaatccacctttgtataaggggtcaaccatcattcttaaaaaacttagtgatttagaacaaaggaaaggaagattttacgggacagcaggttctccaactattgacaatttagaaaatgcctggacgcatttaaccggcagcgctgggacagtgctatcagcttctgggcttggttctatctctttggcgctattggccctttcgaaagctgatgatcatatcttgatgactgatagtgtctacgtgccaacacgtatgctatgtgatggtttattggccaagttcggtgttgaaacggattattatgacccatcaatagggaaggatatagaaaaactagttaagccaaatacaaccgtcattttcctcgaaagcccgggttctgggaccatggaagtacaggatattccagctttggtctctgttgccaaaaagcatgggataaagacaattctagacaacgcatgggcaacgccactcttttttgatgctcatgcgcatggtatcgatatttcggtagaagctgggacaaaatatttgggtggtcattcagatcttcttattggtctggcctccgcaaatgaagaatgttggccgctattacggtcaacttatgatgcaatggcaatgttaccaggtgccgaggactgtcaattagcattgcgaggaatgcgtacattgcacttaagattgaaagaggtagaaagaaaagccctggatttggctgcttggctcggaaatcgagatgaggttgaaaaagtgcttcaccccgcctttgaagattgtcccggacatgaatactgggtttgtgactacaaaggtttttcaggcttattttccattgtccttaaaaatgggttcacaagagctggtttggagaaaatggtagaagggatgaaagttttgcaattgggattttcatggggtggctacgactccttgattacccctgtaaatccttgtaataatagaaaagcttcaacatggccttacaaaggttttgcactaagaatacaagtgggtctcgaagaatttgaagatttaaaaagggattttgagttaggctttgaacgtctcgcagaagaaatttctttgaatcctttacaaatctgattttttttccttttttttttggatttccaagcaatgttgttgagaaatga

SEQ ID NO: 5 - DNA, 68 bp, Artificial Sequence
  Synthetic Primer
aaaaggctcctgatgaaactggagagtctctttgttctgaaatttttaaagtttagcacaccatatag

SEQ ID NO: 6 - DNA, 90 bp, Artificial Sequence
  Synthetic Primer
tccaataacagacagttgcgtagtaataccaaacttcgataactcggtacgatcaatcattttgtttgtttatgtgtgtttattcgaaac

SEQ ID NO: 7 - protein, 401 aa, S. pastorianus
MVDLTELSTFDITTQLTVIGRNPDEQNGFVNPPLYKGSTIIHKKLSDVESMKGRFFYGTAGSPTIANLEDAWTHLTGAAGTVLSPSGLGSITLALMTLAKAGDHILITDSVYVPTRVLCDGLLAKFGIQTEYYDPSVGEDIEKLLKPNTSVIFLESPGSGTMEIQDIPALVSIAKKHGIRTILDNTWATPLFFDAHGHGIDISLEAGTKYLGGHSDFLLGLTSANQECWPLLRSTYDAMAMLPGADDCQLALRGMRTLHLRLKEAERKALDLAAWLGNRDEVEKVLHPAFKDCPGHKYWLRDYKGSSGLFSIVLKDGFTRAGLENMVEKMKIFHLGFSWGGYESLITPVKPAKQRKPHNWPHRGFALRIQVGLEELSDLKKDLELGFKRLAAEISMNPLQL

SEQ ID NO: 8 - protein, 398 aa, S. hominis
MNYNFDEIIDRRYTNAMNVEGYKGYLFGDADTSDLKDNDELIRMWVADMDFGTPEVVLNAIRERLNKKILGYTNVFGSEYYEAFVSWTKKRYGFTFSQEHLVFSHGIVAGLIELVGYICDKDDKALIVTPSYGPFKMACDKNHISTVYSPLINHHGYYEIDFDDVRKKVETENIKLCIFANPHNPTGRVWSEEELATLGQIMKENDVWLISDEIHCDIKRSGQSHIPFAKAVPDYDKIITTMSQSKAFNIAGLMFSNIIIQNESLLKTWNTHHFGTENPLSVVATQAAYEKGEGWLQAMNHYLDDNFNYLADFLEKELPHAEFKIPEATYLAWVDLSYYIKEKDIDESMAKFFIKNAGVIIEGAEQFVHNAEGHIRINIAVPREVMKKGLQKIKAALV

SEQ ID NO: 9 - protein, 399 aa, S. lugdunensis
MTYNFDEIIDRRSTNAMNVEGYLPYLFGNADVSDLQHTDDLIRLWIADMDFATPDVVLNAIRRRLDQKILGYTQVFHVDYYNAFVKWTQSRYGYHFPQEQLVFSHGIVAGLIELVSYICNNEDHALILTPSYGPFKMACDENQVEVHYSPLINEEEYYRIDFDDVERQIVQHNIKLCIFCNPHNPSGRVWSREELERFGNIMKAHDVWLISDEIHCDIMRKGMQHLPFATVLSDYDKVITAMSQSKAFNIAGLMFSNLIIRHKGLLNTWKQQHFGSENPLSIAATQAAYEQGEDWLQAMNTYLDGNFEWLKQFLQQELPNAKFKIPEATYLAWIDLSDYITQQHISEPMAKYFIKRAGVIIEGQEQFVHNADGHIRINLAVPRTVLQQGLTKIKAALMS

SEQ ID NO: 10 - protein, 398 aa, S. devreisei
MSYNFDEIIDRRSTNAMNVEGYKGYLFGDADVLDIEDNDELIRMWVADMDFETPEVVLDAIRDRLDKKILGYTNIFGSDYYNAFVSWTERRYGYTFPQEQLVFSHGIVAGLIELVGYICAKNDKALILTPSYGPFKMACDKNHISTVYSPMINHNGYYEIDFEDVRQKVETENIKLCIFANPHNPTGRVWSEEELRQFGQIMKDNNVWLISDEIHCDIKRSGQTHTPFAKAVPDYDKIITAMSQSKAFNIAGLMFSNIIIPNTRLLNIWKRHHFSTENPLSIVATQAAYEKGEDWLEAMNDYLDGNFQYLADFLERELPHAEFKIPEATYLAWVDLSYYIKDNDINEPLAKYFIKNAGVIIEGQEQFVHNADGHVRINLAVPRKIMQKGLQKIKDALV

SEQ ID NO: 11 - protein, 398 aa, S. petrasii
MTYNFDEIIERRYTNAMNVEGYKGYLFGDADVSDIEDNDELIRMWVADMDFATPEVVLDAIRDRLDKKILGYTNIFGSEYYEAFVSWTKRRFGYTFPQEQLVFSHGIVAGIIELVSYICDEDDKALILTPSYGPFKMACDKNNVSAVYSPLINNNGYYEIDYKDVRKKVETENIKLCIFANPHNPTGRVWSEDELKQLGEIMQDNDVWVISDEIHCDIKRADQTHIPFAKAMPDYDKIITTMSQSKAFNIAGLMFSNIIIPNRRLLKTWKLRHFGTENPLSIAATQAAYEKGEDWLEAMNNYLDDNFKYLAEFLENELPHAKFKIPEATYLAWVDLSYYINQKEIDEPIAKYFIKHAGVIIEGEEQFVHNAEGHVRINIAIPREIMKKGLNKIKEALV

SEQ ID NO: 12 - protein, 398 aa, S. petrasii
MTYNFDEIIERRYTNAMNVEGYKGYLFGDADVSDIEDNDELIRMWVADMDFATPEVVLDAIRDRLDKKILGYTNIFGSEYYEAFVSWTKRRFGYTFPQEQLVFSHGIVAGIIELVSYICDEDDKALILTPSYGPFKMACDKNNVSAVYSPLINNNGYYEIDYKDVRKKVETENIKLCIFANPHNPTGRVWSEDELKQLGEIMQDNDVWVISDEIHCDIKRADQTHIPFAKAVPDYDKIITTMSQSKAFNIAGLMFSNIIIPNRRLLKTWKLRHFGTENPLSIAATQAAYEKGEDWLEAMNHYLDDNFKYLAEFLENELPHAKFKIPEATYLAWVDLSYYINQKEIDESIAKYFIKHAGVIIEGEEQFVHNAEGHVRINIAIPREVMKKGLNKIKEALV

SEQ ID NO: 13 - protein, 355 aa, S. haemolyticus
MWVADMDFATPEVVLDAIRDRLDKKILGYTNIFGTDYYEAFMSWTERRFGYTFPQEHLVFSHGIVAGLIELVSYICDDDDKALIFTPSYGPFKMACDKNNIKTVYSPMINHNGYYEIDFEDVRQKVKTENIKLCIFANPHNPTGRVWSEDELKQLGQIMVDNDVWIISDEIHCDIKRDGQTHVPFAKAVPDYDKIVTAMSQSKAFNIAGLMFSNLIIPNRRLLKTWKLHHFSSENPLSIVATQAAYEKGEDWLAAMNDYLDDNFKYLAQFLEQELPHAEFKIPEATYLAWVDLSYYIKAKHIDEPIAKYFIKHAGVIIEGQEQFVHNAEGHIRINIAVPREIMIKGLQKIKDALV

SEQ ID NO: 14 - protein, 387 aa, S. subtilis
MNFDKREERLGTQSVKWDKTGELFGVTDALPMWVADMDFRAPEAITEALKERLDHGIFGYTTPDQKTKDAVCGWMQNRHGWKVNPESITFSPGVVTALSMAVQAFTEPGDQVVVQPPVYTPFYHMVEKNGRHILHNPLLEKDGAYAIDFEDLETKLSDPSVTLFILCNPHNPSGRSWSREDLLKLGELCLEHGVTVVSDEIHSDLMLYGHKHTPFASLSDDFADISVTCAAPSKTFNIAGLQASAIIIPDRLKRAKFSASLQRNGLGGLNAFAVTAIEAAYSKGGPWLDELITYIEKNMNEAEAFLSTELPKVKMMKPDASYLIWLDFSAYGLSDAELQQRMLKKGKVILEPGTKYGPGGEGFMRLNAGCSLATLQDGLRRIKAALS

SEQ ID NO: 15 - protein, 478 aa, S. cerevisiae
VLSASGLGSISLALLALSKAGDHILMTDSVYVPTRMLCDGLLAKFGVETDYYDPSIGKDIEKLVKPNTTVIFLESPGSGTVLSASGLGSISLALLALSKAGDHILMTDSVYVPTRMLCDGLLAKFGVETDYYDPSIGKDIEKLVKPNTTVIFLESPGSGTMEVQDIPALVSVAKKHGIKTILDNTWATPLFFDAHAHGIDISVEAGTKYLGGHSDLLIGLASANEECWPLLRSTYDAMAMLPGAEDCQLALRGMRTLHLRLKEVERKALDLAAWLGNRDEVEKVLHPAFEDCPGHEYWVRDYKGSSGLFSIVLKNGFTRAGLEKMVEGMKVLQLGFSWGGYDSLITPNPCNNRKASTWPYKGFALRIQVGLEEFEDLKRDFELGFERLAEEISLNPLQIGLEKMVEGMKVLQLGFSWGGYDSLITPNPCNNRKASTWPYKGFALRIQVGLEEFEDLKRDFELGFERLAEEISLNPLQI

(19) United States
(12) Patent Application Publication
(10) Pub. No.: US 2024/0076589 A1
(43) Pub. Date: Mar. 7, 2024

(54) MATERIALS AND METHODS FOR BREWING BEER

(71) Applicant: OMEGA YEAST LABS, LLC, Chicago, IL (US)

(72) Inventors: Laura Burns, Chicago, IL (US); Lance Shaner, Chicago, IL (US)

(73) Assignee: OMEGA YEAST LABS, LLC, Chicago, IL (US)

(21) Appl. No.: 18/272,138

(22) PCT Filed: Feb. 10, 2022
(86) PCT No.: PCT/US22/15947

§ 371 Date: Jul. 13, 2023

Related U.S. Application Data
(60) Provisional application No. 63/147,963, filed on Feb. 10, 2021.
(60) Provisional application No. 63/292,226, filed on Dec. 21, 2021.

(51) Int. Cl. C12C 12/00; C12C 7/04; C12N 9/88

(52) U.S. Cl. CPC C12C 12/006; C12C 7/04; C12N 9/88; C12Y 404/01013

(57) ABSTRACT

Described herein are materials and methods for the conversion of a non-volatile form of 3-sulfanyl-1-hexanol (3SH) to free 3SH during a brewing process.

### CROSS-REFERENCE TO RELATED APPLICATIONS

The present application claims the benefit of priority to U.S. Provisional Application No. 63/147,964, filed Feb. 10, 2021 and U.S. Provisional Application No. 63/292,226, filed Dec. 21, 2021, the disclosures of which are incorporated herein by reference in their entireties.

### INCORPORATION BY REFERENCE OF INFORMATION SUBMITTED ELECTRONICALLY

This application contains, as a separate part of the disclosure, a Sequence Listing in computer readable form (Filename: 56400_Seqlisting.txt; Size: 42,808 bytes; Created: Feb. 9, 2022), which is incorporated by reference in its entirety.

### BACKGROUND

Fruity and floral aromas are in high demand in the beverage industry, and there are continuous efforts to improve the aroma of beer by increasing or diversifying flavor profiles. Thiols, also known as mercaptans, are sulfur-containing organic compounds with a sulfur atom bound to a hydrogen atom. Winemakers identified thiols that contribute to the aroma of wine. One, 4-methyl-4-sulfanylpentan-2-one (4MSP; also known as 4-mercapto-4-methylpentant-2-one (4MMP)), smells and tastes of box tree, black currant, and ribes. Another, 3-sulfanyl-1-hexanol (3SH; also known as 3-mercaptohexanol-1-ol (3MH)) is often described as exotic, smelling of passion fruit, rhubarb and citrus. And the third, 3-sulfanylhexyl acetate (3SHA; also known as 3-mercaptohexyl acetate (3MHA), is reminiscent of passion fruit and guava. These compounds are all prominent in Sauvignon blanc, Riesling, and other wines, although they are not abundant as free form aromatic thiols in grapes. They are formed during fermentation from precursors present in grape must.

Some varieties of hops contain high amounts of the precursor forms of these thiols, but low amounts of the free, aromatic forms that contribute to aroma or flavor of a product. Thus, there remains a need in the art for a means to release these volatile, aromatic thiols from their precursor forms during the fermentation process to maximize the aromatic potential of beer.

### SUMMARY

The disclosure also provides a recombinant yeast comprising a polynucleotide encoding a yeast β-lyase enzyme Irc7 operably linked to a heterologous promoter, wherein the β-lyase enzyme comprises an amino acid sequence at least 95% identical to the amino acid sequence set forth in SEQ ID NO: 1.

The disclosure provides a recombinant Saccharomyces spp comprising a polynucleotide encoding an yeast β-lyase enzyme IRC7 operably linked to a heterologous promoter, wherein the β-lyase enzyme comprises an amino acid sequence at least 95% identical to the amino acid sequence set forth in SEQ ID NO: 1.

The disclosure also provides a recombinant yeast comprising a polynucleotide encoding a cysteine-thiol lyase operably linked to a heterologous promoter. In some embodiments, the cysteine-thiol lyase is PatB. In some embodiments, the PatB is from species of Staphylococcus. In some embodiments, the PatB is from S. lugdunensis, S. devriesei, S. hominis, S. haemolyticus, S. petrasii, or B. subtilis. In some embodiments, the PatB is from S. petrasii croceilyticus or S. petrasii petrasii. In some embodiments, the PatB comprises an amino acid sequence at least 80% identical to any one of SEQ ID NOs: 8-14. In some embodiments, the PatB comprises an amino acid sequence set forth in any one of SEQ ID NOs: 8-14.

In some embodiments, the heterologous promoter is TDH3, TDH2, CCW12, PGK1, ADH1, ADH2, CYC1, HHF1, HHF2, TEF1, TEF2, HTB2, PAB1, ALD6, RNR1, RNR2, POP6, RAD27, PSP2, REV1, MFA1, MFa2, GAL1, CUP1, MET25, ICL1, ICL2, GAL3, HXT1, HXT2, MAL11, MAL31, MAL32, MAL33, MRK1, or SUC2 promoter. In some embodiments, the recombinant Saccharomyces spp is S. cerevisiae or S. pastorianus. In some embodiments, the β-lyase enzyme comprises the amino acid sequence set forth in SEQ ID NO: 1. In some embodiments, the β-lyase enzyme does not comprise the amino acid sequence set forth in SEQ ID NO: 3.

The disclosure also provides a method of converting a non-volatile form of 3-sulfanyl-1-hexanol (3SH) to free 3SH during a brewing process, the method comprising contacting cooled wort with the recombinant Saccharomyces described herein under conditions and for a time sufficient to convert non-volatile form of 3SH to free 3SH. In some embodiments, the non-volatile form of 3SH is glutathione-bound 3SH or cysteine-bound-3SH or a combination thereof. In some embodiments, the method results in a 3-fold increase in free 3SH in wort fermented with the recombinant Saccharomyces compared to wort fermented with non-modified Saccharomyces. In some embodiments, the wort comprises at least 60 ng/L free 3SH after the contacting step.

The method optionally comprises adding hops to cooled wort during the contacting step. Exemplary hops for use in the methods described herein include, but are not limited to, Cascade, Calypso, Hallertau Tradition, Hallertau Perle, Triple Pearl, Nugget, Saaz, Columbus/CTZ, Chinook, Nelson Sauvin, Hallertau Blanc or Simcoe. In some embodiments, the hops contain at least 400 m/kg of cysteine-bound 3SH.

The disclosure also provides a method of converting a non-volatile form of 3-sulfanyl-1-hexanol (3SH) to free 3SH during a brewing process, the method comprising (a) a mash hopping step comprising adding a plant material comprising a non-volatile form of 3SH to grist to produce wort; (b) boiling the wort produced by (a); (c) cooling the wort; and (d) contacting the cooled wort with a recombinant yeast described herein for a time sufficient to convert a non-volatile form of 3SH to free 3SH. In some embodiments, the recombinant yeast is a recombinant Saccharomyces comprising a polynucleotide encoding a β-lyase enzyme Irc7 operably linked to a heterologous promoter. In some embodiments, the recombinant yeast is a recombinant Saccharomyces spp. comprising a polynucleotide encoding a cysteine-thiol lyase operably linked to a heterologous promoter. In some embodiments, the non-volatile form of 3SH is glutathione-bound-3SH or cysteine-bound-3SH, or a combination thereof. In some embodiments, the method results in a 6-fold increase in free 3SH in wort fermented with the recombinant yeast compared to wort fermented with non-modified yeast. In some embodiments, the wort comprises at least 60 ng/L free 3SH after the contacting step.

In some embodiments, the plant material comprises a non-volatile form of 3SH is hops. In some embodiments, the hops contain at least 400 μg/kg of cysteine-bound 3SH. In some embodiments, the hops contain at least 5000 μg/kg glutathione-3SH. In some embodiments, the hops contain at least 400 μg/kg of cysteine-bound 3SH and at least 5000 μg/kg glutathione-3SH. In some embodiments, the hops are Cascade, Calypso, Hallertau Tradition, Hallertau Perle, Triple Pearl, Nugget, Saaz, Columbus/CTZ, Chinook, Nelson Sauvin, Hallertau Blanc or Simcoe hops.

In some embodiments, the plant material comprises a non-volatile form of 3SH is a grape-derived product. In some embodiments, the grape derived product is crushed grapes or grape flour. In some embodiments, the grape-derived product is obtained from a white grape, a red grape, or combinations thereof. Exemplary white grape varieties include, but are not limited to, Sauvignon Blanc, Chardonnay, Chenin Blanc, Colombard, Gewurztraminer, Gros Manseng, Koshu, Maccabeo, Muscat, Petit Manseng, Pinot Blanc, Pinot Gris, Riesling, Scheurebe, Semillon, Sylvaner, and Tokay. Exemplary red grape varieties include, but are not limited to, Cabernet Franc, Cabernet Sauvignon, Grenache, Merlot, and Pinot Noir.

In some embodiments, the mash hopping step comprises adding less than 100 grams of the plant material per 1 kg of grist.

In some embodiments, the mash hopping step comprises adding both hops and a grape-derived product to the grist.

In some embodiments, the β-lyase is a bacterial β-lyase or a fungal β-lyase. Exemplary bacterial β-lyases for use according to the disclosure are from bacteria including, but not limited to, Eschericia sp.; Thermoanaerobacter sp.; Symbiobacterium sp.; Photobacterium sp.; Haemophilus sp.; Vibrio sp.; Proteus sp.; Halobacterium sp.; Desulfitobacterium sp.; and Treponema sp. In some embodiments, the bacterial β-lyase is E. coli TNaA.

In some embodiments, the fungal β-lyase is a yeast β-lyase. Exemplary fungal β-lyases for use according to the disclosure are from Saccharomycotina, Taphrinomycotina, and Schizosaccharomycetes. In some embodiments, the yeast β-lyase is from Saccharomyces. In some embodiments, the yeast β-lyase is Irc7. In some embodiments, the Irc7 comprises an amino acid sequence at least 95% (e.g., at least 96%, at least 97%, at least 98%, at least 99% or more) identical to the amino acid sequence set forth in SEQ ID NO: 1. In some embodiments, the Irc7 comprises the amino acid sequence set forth in SEQ ID NO: 1.

In some embodiments, the mash hopping step comprises a protein rest before the boiling step. In some embodiments, the protein rest comprises maintaining the mash at a temperature of less than 140° F. for at least five minutes before the boiling step. In some embodiments, the protein rest comprises maintaining the mash at a temperature between 100° F. and 140° F. for at least one hour before the boiling step. In some embodiments, the mash hopping step further comprises a saccharification rest after the protein rest and before the boiling step.

In some embodiments, the method further comprises contacting the cooled wort with hops to produce an admixture and contacting the admixture with the recombinant yeast. In some embodiments, the recombinant yeast is S. cerevisiae or S. pastorianus.

In some embodiments, the contacting step (d) occurs in a fermenter at a temperature ranging from 45° F.-100° F.

### BRIEF DESCRIPTION OF THE FIGURES

FIG. 1 is an alignment of IRC7 alleles described in Example 1.

FIG. 2 is a bar graph showing that free 3SH levels are increased in beer fermented with OYL-088-TDH3-IRC7 compared to the unmodified OYL-088 strain.

### DETAILED DESCRIPTION

Many plant derived products contain volatile thiols bound as cysteine S-conjugate precursors, and conversion of the non-volatile precursor to a volatile thiol product contributes to the aroma of a food or drink product. Volatile thiols are responsible for imparting aromas such as box tree, passionfruit, grapefruit, gooseberry, and guava to a fermented beverage, such as beer.

The present disclosure is based, in part, on the discovery that yeast (e.g., Saccharomyces spp.) that have been modified to overexpress a yeast β-lyase enzyme (or a cysteine-thiol lyase) triggers the conversion of available non-volatile thiols (e.g., glutathione bound- or cysteine-bound thiols) in plant material to the more desirable aromatic (i.e., volatile or free) form during the fermentation step of a brewing process.

The present disclosure is also based, in part, on the discovery that modifying the conventional brewing method to include a mashing step comprising adding a plant material comprising a non-volatile thiol of interest to grist to produce wort, and subsequently contacting the wort with a modified Saccharomyces spp that overexpresses a β-lyase enzyme (or a cysteine-thiol lyase), results in higher rates of conversion of available non-volatile thiols to the volatile, aromatic form.

### Recombinant Yeast.

In one aspect, described herein is a recombinant yeast comprising a polynucleotides encoding a β-lyase (or cysteine-thiol lyase) enzyme operably linked to a heterologous promoter. The term “heterologous promoter” as used herein refers to a promoter that is non-native to the β-lyase (or cysteine-thiol lyase) enzyme.

In some embodiments, the yeast belongs to a non-Saccharomyces genus. In some embodiments, the yeast belongs to the genus Kloeckera, Candida, Starmerella, Hanseniaspora, Kluyveromyces/Lachance, Metschnikowia, Saccharomycodes, Zygosaccharomyce, Dekkera (also referred to as Brettanomyces), Wickerhamomyces, or Torulaspora. In some embodiments, the yeast is Hanseniaspora uvarum, Hanseniaspora guillermondii, Hanseniaspora vinae, Metschnikowia pulcherrima, Kluyveromyces/Lachancea thermotolerans, Starmerella bacillaris (previously referred to as Candida stellatal or Candida zemplinina), Saccharomycodes ludwigii, Zygosaccharomyces rouxii, Dekkera bruxellensis, Dekkera anomala, Brettanomyces custersianus, Brettanomyces naardenensis, Brettanomyces nanus, Wickerhamomyces anomalus, or Torulaspora delbrueckii.

In another aspect, described herein is a recombinant Saccharomyces spp. comprising a polynucleotide encoding a β-lyase (or a cysteine-thiol lyase) enzyme operably linked to a heterologous promoter.

In various aspects, the recombinant Saccharomyces is S. cerevisiae or S. pastorianus.

β-lyase is an enzyme responsible for the release of volatile sulfur compounds called polyfunctional thiols, or mercaptans, which are usually associated with tropical aroma. Exemplary β-lyase enzymes for use in accordance with the present disclosure include, but are not limited to, those described in International Publication No. WO 2007/095682, the disclosure of which is incorporated herein by reference in its entirety. In some embodiments, the β-lyase is a bacterial β-lyase, such as a β-lyase from Eschericia sp.; Thermoanaerobacter sp.; Symbiobacterium sp.; Photobacterium sp.; Haemophilus sp.; Vibrio sp.; Proteus sp; Halobacterium sp.; Desulfitobacterium sp; or Treponema sp. In some embodiments, the β-lyase is tryptophanase (E. coli) (UniProt Accession No. P0A853).

In some embodiments, the β-lyase is a fungal β-lyase, such as a yeast β-lyase from Saccharomyces cerevisiae (all strains), Saccharomyces bayanus and species of Brettanomyces and Dekkera; Candida; Cryptococcus; Debaryomyces; Hanseniaspora, Kloeckera; Kluyveromyces; Metschnikowia; Pichia; Rhodotorula; Saccharomyces; Saccharomycodes; Schizosaccharomyces; or Zygosaccharomyces. In some embodiments, the β-lyase is IRC7. In some embodiments, β-lyase is comprises an amino acid sequence at least 95% identical (e.g., 95%, 96%, 97%, 98%, 99% or more) identical to the amino acid sequence set forth in SEQ ID NO: 1.

In some embodiments, the cysteine-thiol lyase has a Kcat/Km for cystathionine of less than or equal to 0.7×10² min⁻¹ mM⁻¹ (e.g., 0.7×10² min⁻¹ mM⁻¹, 0.6×10² min⁻¹ mM⁻¹, 0.5×10² min⁻¹ mM⁻¹, 0.4×10² min⁻¹ mM⁻¹, 0.3×10² min⁻¹ mM⁻¹, 0.2×10² min⁻¹ mM⁻¹, 0.1×10² min⁻¹ mM⁻¹ or less). In some embodiments, the cysteine-thiol lyase Kcat/Km for Cys-3M3SH of greater than or equal to 3×10² min⁻¹ mM⁻¹ (e.g., 3×10² min⁻¹ mM⁻¹, 3.5×10² min⁻¹ mM⁻¹, 4×10² min⁻¹ mM⁻¹, 4.5×10² min⁻¹ mM⁻¹, 5×10² min⁻¹ mM⁻¹, 5.5×10² min⁻¹ mM⁻¹, 6×10² min⁻¹ mM⁻¹, 6.5×10² min⁻¹ mM⁻¹, 7×10² min⁻¹ mM⁻¹, 7.5×10² min⁻¹ mM⁻¹, 8×10² min⁻¹ mM⁻¹, 8.5×10² min⁻¹ mM⁻¹, 9×10² min⁻¹ mM⁻¹, 9.5×10² min⁻¹ mM⁻¹ or higher).

In some embodiments, the cysteine-thiol lyase is PatB. In some embodiments, the PatB is from the Staphylococcus genus. In some embodiments, the PatB is from S. lugdunensis (SEQ ID NO: 9), S. devriesei (SEQ ID NO: 10), S. hominis (SEQ ID NO: 8), S. haemolyticus (SEQ ID NO: 13), S. petrasii (SEQ ID NO: 11 or SEQ ID NO:12), or B. subtilis (SEQ ID NO: 14)

In another exemplary aspect, the disclosure provides a recombinant yeast comprising a polynucleotide encoding PatB operably linked to a heterologous promoter, wherein the PatB comprises an amino acid sequence that is at least 80% identical (e.g., 80%, 81%, 82%, 83%, 84%, 85%, 86%, 97%, 88%, 89%, 90%, 91%, 92%, 93%, 94%, 95%, 96%, 97%, 98%, 99% or more identical) to the amino acid sequence set forth in SEQ ID NO: 8. In some embodiments, the PatB comprises an amino acid sequence set forth in SEQ ID NO: 8.

In another exemplary aspect, the disclosure provides a recombinant yeast comprising a polynucleotide encoding PatB operably linked to a heterologous promoter, wherein the PatB comprises an amino acid sequence that is at least 80% identical (e.g., 80%, 81%, 82%, 83%, 84%, 85%, 86%, 97%, 88%, 89%, 90%, 91%, 92%, 93%, 94%, 95%, 96%, 97%, 98%, 99% or more identical) to the amino acid sequence set forth in SEQ ID NO: 9. In some embodiments, the PatB comprises an amino acid sequence set forth in SEQ ID NO: 9.

In another exemplary aspect, the disclosure provides a recombinant yeast comprising a polynucleotide encoding PatB operably linked to a heterologous promoter, wherein the PatB comprises an amino acid sequence that is at least 80% identical (e.g., 80%, 81%, 82%, 83%, 84%, 85%, 86%, 97%, 88%, 89%, 90%, 91%, 92%, 93%, 94%, 95%, 96%, 97%, 98%, 99% or more identical) to the amino acid sequence set forth in SEQ ID NO: 10. In some embodiments, the PatB comprises an amino acid sequence set forth in SEQ ID NO: 10.

In another exemplary aspect, the disclosure provides a recombinant yeast comprising a polynucleotide encoding PatB operably linked to a heterologous promoter, wherein the PatB comprises an amino acid sequence that is at least 80% identical (e.g., 80%, 81%, 82%, 83%, 84%, 85%, 86%, 97%, 88%, 89%, 90%, 91%, 92%, 93%, 94%, 95%, 96%, 97%, 98%, 99% or more identical) to the amino acid sequence set forth in SEQ ID NO: 11. In some embodiments, the PatB comprises an amino acid sequence set forth in SEQ ID NO: 11.

In another exemplary aspect, the disclosure provides a recombinant yeast comprising a polynucleotide encoding PatB operably linked to a heterologous promoter, wherein the PatB comprises an amino acid sequence that is at least 80% identical (e.g., 80%, 81%, 82%, 83%, 84%, 85%, 86%, 97%, 88%, 89%, 90%, 91%, 92%, 93%, 94%, 95%, 96%, 97%, 98%, 99% or more identical) to the amino acid sequence set forth in SEQ ID NO: 12. In some embodiments, the PatB comprises an amino acid sequence set forth in SEQ ID NO: 12.

In another exemplary aspect, the disclosure provides a recombinant yeast comprising a polynucleotide encoding PatB operably linked to a heterologous promoter, wherein the PatB comprises an amino acid sequence that is at least 80% identical (e.g., 80%, 81%, 82%, 83%, 84%, 85%, 86%, 97%, 88%, 89%, 90%, 91%, 92%, 93%, 94%, 95%, 96%, 97%, 98%, 99% or more identical) to the amino acid sequence set forth in SEQ ID NO: 13. In some embodiments, the PatB comprises an amino acid sequence set forth in SEQ ID NO: 13.

In another exemplary aspect, the disclosure provides a recombinant yeast comprising a polynucleotide encoding PatB operably linked to a heterologous promoter, wherein the PatB comprises an amino acid sequence that is at least 80% identical (e.g., 80%, 81%, 82%, 83%, 84%, 85%, 86%, 97%, 88%, 89%, 90%, 91%, 92%, 93%, 94%, 95%, 96%, 97%, 98%, 99% or more identical) to the amino acid sequence set forth in SEQ ID NO: 14. In some embodiments, the PatB comprises an amino acid sequence set forth in SEQ ID NO: 14.

In some embodiments, the recombinant yeast comprising a polynucleotide encoding a cysteine-thiol lyase (e.g., PatB) promotes the release of 4-methyl-4-sulfanylpentan-2-one (4MSP) that is about 2-fold greater than a recombinant yeast comprising a polynucleotide encoding a TnaA enzyme.

The terms “operably-linked” or “functionally-linked” refer to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

The term “promoter” refers to a nucleotide sequence, usually upstream (5′) to its coding sequence, which controls the expression of the coding sequence by providing the recognition site for RNA polymerase and other factors required for proper transcription. “Promoter” includes a minimal promoter that is a short DNA sequence comprised, in some cases, of a TATA box and other sequences that serve to specify the site of transcription initiation, to which regulatory elements are added for enhancement of expression. “Promoter” also refers to a nucleotide sequence that includes a minimal promoter plus regulatory elements and that is capable of controlling the expression of a coding sequence or functional RNA. This type of promoter sequence consists of proximal and more distal upstream elements, the latter elements often referred to as enhancers. Accordingly, an “enhancer” is a DNA sequence, which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements, derived from different promoters found in nature, or even be comprised of synthetic DNA segments.

A promoter may also contain DNA sequences that are involved in the binding of protein factors, which control the effectiveness of transcription initiation in response to physiological or developmental conditions. The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

Exemplary promoters include, but are not limited to, TDH3, TDH2, CCW12, PGK1, ADH1, ADH2, CYC1, HHF1, HHF2, TEF1, TEF2, HTB2, PAB1, ALD6, RNR1, RNR2, POP6, RAD27, PSP2, REV1, MFA1, MFa2, GAL1, CUP1, MET25, ICL1, ICL2, GAL3, HXT1, HXT2, MAL11, MAL31, MAL32, MAL33, MRK1, and SUC2 promoters. In some embodiments, the promoter is the TDH3 promoter.

In an exemplary aspect, the disclosure provides a recombinant yeast (e.g., Saccharomyces spp.) comprising a polynucleotide encoding a yeast β-lyase enzyme Irc7 operably linked to a heterologous promoter, wherein the β-lyase enzyme comprises an amino acid sequence at least 95% identical (e.g., 95%, 96%, 97%, 98%, 99% or more identical) to the amino acid sequence set forth in SEQ ID NO: 1. In some embodiments, the β-lyase enzyme comprises an amino acid sequence set forth in SEQ ID NO: 1. In some embodiments, the β-lyase enzyme does not comprise the amino acid sequence set forth in SEQ ID NO: 3.

Techniques for the recombinant expression of enzymes in a cell and genetic modification of a recombinant yeast cell are well known to those skilled in the art. Typically such techniques involve transformation of a cell with nucleic acid construct comprising the relevant sequence. Such methods are, for example, known from standard handbooks, such as Sambrook and Russel (2001) “Molecular Cloning: A Laboratory Manual (3rd edition), Cold Spring Harbor Laboratory, Cold Spring Harbor Laboratory Press, or F. Ausubel et al., eds., “Current protocols in molecular biology”, Green Publishing and Wiley Interscience, New York (1987). Methods for transformation and genetic modification of fungal host cells are described in, e.g., European Application No. EP-A-0635574, International Patent Publication No. WO 98/46772, International Patent Publication No. WO 99/60102, International Patent Publication No. WO 00/37671, International Patent Publication No. WO 90/14423, European Application No. EP-A-0481008, European Application No. EP-A-0635574 and U.S. Pat. No. 6,265,186, the disclosures of which are incorporated herein by reference in their entireties.

### Methods

The disclosure provides a method of converting a non-volatile form of 3-sulfanyl-1-hexanol (3SH) to free 3SH during a brewing process. In one aspect, the method comprises contacting a fermentable sugar source (e.g., cooled wort) with the recombinant yeast described herein (e.g., a recombinant Saccharomyces spp. comprising a polynucleotide encoding a yeast β-lyase enzyme IRC7 comprising an amino acid sequence at least 95% identical to the amino acid sequence set forth in SEQ ID NO: 1, operably linked to a heterologous promoter; (or a recombinant Saccharomyces spp. comprising a polynucleotide encoding a cysteine-thiol lyase PatB, operably linked to a heterologous promoter), under conditions and for a time sufficient to convert non-volatile form of 3SH to free 3SH.

In another aspect, the disclosure provides a method of converting a non-volatile form of 3-sulfanyl-1-hexanol (3SH) to free 3SH during a brewing process., the method comprising contacting a fermentable sugar source with a cysteine-thiol lyase, under conditions and for a time sufficient to convert non-volatile form of 3SH to free 3SH. In some embodiments, the cysteine-thiol lyase is purified before the contacting step. Purification of cysteine-thiol lyases can be performed as described in Rudden et al., Scientific Reports, 10:12500, 2020, the disclosure of which is incorporated herein by reference.

In some embodiments, the fermentable sugar source is wort, grains/cereals, fruit juice (e.g., grape juice, apple juice/cider), honey, cane sugar, rice, or koji.

In some embodiments, the non-volatile form of 3SH is glutathione-bound 3SH or cysteine-bound-3SH, or a combination thereof. In some embodiments, the method results in a 3-fold increase in free 3SH in the fermentable sugar source (e.g., wort) fermented with the recombinant yeast (e.g., Saccharomyces) compared to the fermentable sugar source (e.g.,wort) fermented with non-modified yeast (e.g.,Saccharomyces).

In some embodiments, the method results in a 3-fold increase in free 3SH in the fermentable sugar source (e.g., wort) with the cysteine-thiol lyase compared to the fermentable sugar source (e.g.,wort) without the cysteine-thiol lyase.

In some embodiments, the cysteine-thiol lyase is PatB. In some embodiments, the PatB is from species of Staphylococcus. In some embodiments, the PatB is from S. lugdunensis (SEQ ID NO: 9), S. devriesei (SEQ ID NO: 10), S. hominis (SEQ ID NO: 8), S. haemolyticus (SEQ ID NO: 13), S. petrasii (SEQ ID NO: 11 or SEQ ID NO:12), or B. subtilis (SEQ ID NO: 14)

In some embodiments, the PatB comprises an amino acid sequence that is at least 80% identical (e.g., 80%, 81%, 82%, 83%, 84%, 85%, 86%, 97%, 88%, 89%, 90%, 91%, 92%, 93%, 94%, 95%, 96%, 97%, 98%, 99% or more identical) to the amino acid sequence set forth in SEQ ID NO: 8. In some embodiments, the PatB comprises an amino acid sequence set forth in SEQ ID NO: 8.

In some embodiments, the PatB comprises an amino acid sequence that is at least 80% identical (e.g., 80%, 81%, 82%, 83%, 84%, 85%, 86%, 97%, 88%, 89%, 90%, 91%, 92%, 93%, 94%, 95%, 96%, 97%, 98%, 99% or more identical) to the amino acid sequence set forth in SEQ ID NO: 9. In some embodiments, the PatB comprises an amino acid sequence set forth in SEQ ID NO: 9.

In some embodiments, the PatB comprises an amino acid sequence that is at least 80% identical (e.g., 80%, 81%, 82%, 83%, 84%, 85%, 86%, 97%, 88%, 89%, 90%, 91%, 92%, 93%, 94%, 95%, 96%, 97%, 98%, 99% or more identical) to the amino acid sequence set forth in SEQ ID NO: 10. In some embodiments, the PatB comprises an amino acid sequence set forth in SEQ ID NO: 10.

In some embodiments, the PatB comprises an amino acid sequence that is at least 80% identical (e.g., 80%, 81%, 82%, 83%, 84%, 85%, 86%, 97%, 88%, 89%, 90%, 91%, 92%, 93%, 94%, 95%, 96%, 97%, 98%, 99% or more identical) to the amino acid sequence set forth in SEQ ID NO: 11. In some embodiments, the PatB comprises an amino acid sequence set forth in SEQ ID NO: 11.

In some embodiments, the PatB comprises an amino acid sequence that is at least 80% identical (e.g., 80%, 81%, 82%, 83%, 84%, 85%, 86%, 97%, 88%, 89%, 90%, 91%, 92%, 93%, 94%, 95%, 96%, 97%, 98%, 99% or more identical) to the amino acid sequence set forth in SEQ ID NO: 12. In some embodiments, the PatB comprises an amino acid sequence set forth in SEQ ID NO: 12.

In some embodiments, the PatB comprises an amino acid sequence that is at least 80% identical (e.g., 80%, 81%, 82%, 83%, 84%, 85%, 86%, 97%, 88%, 89%, 90%, 91%, 92%, 93%, 94%, 95%, 96%, 97%, 98%, 99% or more identical) to the amino acid sequence set forth in SEQ ID NO: 13. In some embodiments, the PatB comprises an amino acid sequence set forth in SEQ ID NO: 13.

In some embodiments, the PatB comprises an amino acid sequence that is at least 80% identical (e.g., 80%, 81%, 82%, 83%, 84%, 85%, 86%, 97%, 88%, 89%, 90%, 91%, 92%, 93%, 94%, 95%, 96%, 97%, 98%, 99% or more identical) to the amino acid sequence set forth in SEQ ID NO: 14. In some embodiments, the PatB comprises an amino acid sequence set forth in SEQ ID NO: 14.

The disclosure also provides a method of converting of a non-volatile form of 3-sulfanyl-1-hexanol (3SH) to free 3SH during a brewing process, wherein the method comprises (a) a mash hopping step comprising adding a plant material comprising a non-volatile form of 3SH to grist to produce wort; (b) boiling the wort produced by (a); (c) cooling the wort; and (d) contacting the cooled wort with a recombinant Saccharomyces described herein under conditions and for a time sufficient to convert a non-volatile form of 3SH to free 3SH. In some embodiments, the non-volatile form of 3SH is glutathione-bound-3SH or cysteine-bound-3SH, and combinations thereof.

In some embodiments, methods of converting a non-volatile form of 3-sulfanyl-1-hexanol (3SH) to free 3SH during a brewing process can be performed in the absence of a recombinant yeast comprising a polynucleotide a cysteine-thiol lyase operably linked to a heterologous promoter. In this regard, the disclosure also provides a method of converting of a non-volatile form of 3-sulfanyl-1-hexanol (3SH) to free 3SH during a brewing process, wherein the method comprises (a) a mash hopping step comprising adding a plant material comprising a non-volatile form of 3SH to grist to produce wort; (b) boiling the wort produced by (a); (c) cooling the wort; and (d) contacting the cooled wort with a purified cysteine-thyol lyase under conditions and for a time sufficient to convert a non-volatile form of 3SH to free 3SH. In some embodiments, the non-volatile form of 3SH is glutathione-bound-3SH or cysteine-bound-3SH, and combinations thereof.

The phrase “plant material comprising a non-volatile form of 3SH” refers to any plant (or plant part) containing glutathione-bound-3SH, cysteine-bound-3SH, or combinations thereof. The term “plant parts” encompasses all components of a plant including seeds, shoots, stems, leaves, roots, flowers, and plant tissues. In some embodiments, the plant material has been processed prior to being added to the grist. Exemplary methods of processing plant material include, but are not limited to, crushing, pressing, slicing, blending, juicing, rolling, pulverizing or grinding the plant material.

In some embodiments, the plant material comprising a non-volatile form of 3SH is hops. Hops suitable for use in the methods described herein include, but are not limited to, Amarillo, Apollo, Cascade, Centennial, Chinook, Citra, Cluster, Columbus, Crystal, Eroica, Galena, Glacier, Greenburg, Horizon, Liberty, Millenium, Mount Hood, Mount Rainier, Newport, Nugget, Palisade, Santiam, Simcoe, Sterling, Summit, Tomahawk, Ultra, Vanguard, Warrior, Willamette, Zeus, Admiral, Brewer's Gold, Bullion, Challenger, First Gold, Fuggles, Goldings, Herald, Northdown, Northern Brewer, Phoenix, Pilot, Pioneer, Progress, Target, Whitbread Golding Variety (WGV), Hallertau, Hersbrucker, Saaz, Tettnang, Spalt, Feux-Coeur Francais, Galaxy, Green Bullet, Motueka, Nelson Sauvin, Pacific Gem, Pacific Jade, Pacifica, Pride of Ringwood, Riwaka, Southern Cross, Lublin, Magnum, Perle, Polnischer Lublin, Saphir, Satus, Select, Strisselspalt, Styrian Goldings, Tardif de Bourgogne, Tradition, Bravo, Calypso, Chelan, Comet, El Dorado, San Juan Ruby Red, Satus, Sonnet Golding, Super Galena, Tillicum, Bramling Cross, Pilgrim. Hallertauer Herkules, Hallertauer Magnum, Hallertauer Taurus, Merkur, Opal, Smaragd, Halleratau Aroma, Kohatu, Rakau, Stella, Sticklebract, Summer Saaz, Super Alpha, Super Pride, Topaz, Wai-iti, Bor, Junga, Marynka, Premiant, Sladek, Styrian Atlas, Styrian Aurora, Styrian Bobek, Styrian Celeia, Sybilla, and Sorachi Ace hops. In some embodiments, the hops are Cascade, Calypso, Hallertau Tradition, Hallertau Perle, Triple Pearl, Nugget, Saaz, Columbus/CTZ, Chinook, Nelson Sauvin, Hallertau Blanc, and/or Simcoe hops.

In some embodiments, the hops contain at least about 400 μg/kg of cysteine-bound 3SH. For example, in some embodiments, the hops contain at least about 400 μg/kg, at least about 450 μg/kg, at least about 500 μg/kg, at least about 550 μg/kg, at least about 600 μg/kg, at least about 650 μg/kg, at least about 700 μg/kg, at least about 750 μg/kg, at least about 800 μg/kg, at least about 850 μg/kg, at least about 900 μg/kg, at least about 950 μg/kg, or at least about 1000 μg/kg cysteine-bound-3SH. In some embodiments, the hops contain an amount of cysteine-bound-3SH ranging from about 400 μg/kg to about 1000 μg/kg, or from about 500 μg/kg to about 900 μg/kg, or from about 600 μg/kg to about 800 μg/kg, or from about 400 μg/kg to about 600 μg/kg.

In some embodiments, the hops contain at least about 5000 μg/kg glutathione-3SH. For example, in some embodiments, the hops contain at least about 5000 μg/kg, at least about 5500 μg/kg, at least about 6000 μg/kg, at least about 6500 μg/kg, at least about 7000 μg/kg, at least about 7500 μg/kg, at least about 5000 μg/kg, at least about 8500 μg/kg, at least about 9000 μg/kg, at least about 9500 μg/kg, or at least about 10,000 μg/kg, at least about 10,500 μg/kg, at least about 11,000 μg/kg, at least about 11,500 μg/kg, at least about 12,000 μg/kg, at least about 12,500 μg/kg, at least about 13,000 μg/kg, at least about 13,500 μg/kg, at least about 14,000 μg/kg, at least 14,500 μg/kg, at last about 15,000 μg/kg, at least about 15,500 μg/kg, at least about 16,000 μg/kg, at least about 16,500 μg/kg, at least about 17,000 μg/kg, at least about 17,500 μg/kg, at least about 18,000 μg/kg, at least about 18,500 μg/kg, at least about 19,000 μg/kg, at least about 19,500 μg/kg, or at least about 20,000 μg/kg glutathione-bound-3SH. In some embodiments, the hops contain an amount of glutathione-bound-3SH ranging from about 5000 μg/kg to about 1000 μg/kg, or from about 5500 μg/kg to about 9000 μg/kg, or from about 6000 μg/kg to about 8000 μg/kg, or from about 4000 μg/kg to about 6000 μg/kg, or from about 5000 μg/kg to about 8000 μg/kg, or from about 8000 μg/kg to about 12,000 μg/kg, or from about 10,000 μg/kg to about 20,000 μg/kg, or from about 15,000 μg/kg to about 20,000 μg/kg.

In some embodiments, plant material comprising a non-volatile form of 3SH is grape-derived product. Suitable grape derived products include, e.g., crushed grapes and grape flour. The grape-derived product may be obtained from a white grape, a red grape, or combinations thereof. Exemplary white grape varieties include, but are not limited to, Sauvignon Blanc, Chardonnay, Chenin Blanc, Colombard, Gewurztraminer, Gros Manseng, Koshu, Maccabeo, Muscat, Petit Manseng, Pinot Blanc, Pinot Gris, Riesling, Scheurebe, Semillon, Sylvaner, and Tokay. Exemplary red grape varieties include, but are not limited to, Cabernet Franc, Cabernet Sauvignon, Grenache, Merlot, and Pinot Noir.

In some embodiments, the grape-derived product contains at least about 400 μg/kg of cysteine-bound 3SH. For example, in some embodiments, the grape-derived product contains at least about 400 μg/kg, at least about 450 μg/kg, at least about 500 μg/kg, at least about 550 μg/kg, at least about 600 μg/kg, at least about 650 μg/kg, at least about 700 μg/kg, at least about 750 μg/kg, at least about 800 μg/kg, at least about 850 μg/kg, at least about 900 μg/kg, at least about 950 μg/kg, or at least about 1000 μg/kg, or at least about 1500 μg/kg, or at least about 2000 μg/kg, or at least about 2500 μg/kg, or at least about 3000 μg/kg, or at least 3500 μg/kg, or at least about 4000 μg/kg, or at least about 4500 μg/kg, or at least about 5000 μg/kg, at least about 5500 μg/kg, at least about 6000 μg/kg, at least about 6500 μg/kg, at least about 7000 μg/kg, at least about 7500 μg/kg, at least about 8000 μg/kg, at least about 8500 μg/kg, at least about 9000 μg/kg, at least about 9500 μg/kg, or at least about 10,000 μg/kg, at least about 10,500 μg/kg, at least about 11,000 μg/kg, at least about 11,500 μg/kg, at least about 12,000 μg/kg, at least about 12,500 μg/kg, at least about 13,000 μg/kg, at least about 13,500 μg/kg, at least about 14,000 μg/kg, at least 14,500 μg/kg, at last about 15,000 μg/kg, at least about 15,500 μg/kg, at least about 16,000 μg/kg, at least about 16,500 μg/kg, at least about 17,000 μg/kg, at least about 17,500 μg/kg, at least about 18,000 μg/kg, at least about 18,500 μg/kg, at least about 19,000 μg/kg, at least about 19,500 μg/kg, or at least about 20,000 μg/kg, or at least 20,500 μg/kg, or at least 21,000 μg/kg, or at least 21,500 μg/kg, or at least 22,000 μg/kg, or at least 22,500 μg/kg, or at least 23,000 μg/kg, or at least 23,500 μg/kg, or at least 24,000 μg/kg, or at least 24,500 μg/kg, or at least 25,000 μg/kg, or at least 25,500 μg/kg, or at least 26,000 μg/kg, or at least 26,500 μg/kg, or at least 27,000 μg/kg, or at least 27,500 μg/kg, or at least 28,000 μg/kg, or at least 28,500 μg/kg, or at least 29,000 μg/kg, or at least 29,500 μg/kg, or at least 30,000 μg/kg, or at least 30,500 μg/kg, or at least 31,000 μg/kg, or at least 31,500 μg/kg, or at least 32,000 μg/kg, or at least 33,000 μg/kg, or at least 33,500 μg/kg, or at least 34,000 μg/kg, or at least 34,500 μg/kg, or at least 35,000 μg/kg, or at least 35,500 μg/kg, or at least 36,000 μg/kg cysteine-bound-3SH. In some embodiments, the grape derived product contains an amount of cysteine-bound-3SH ranging from about 400 μg/kg to about 1000 μg/kg, or from about 500 μg/kg to about 900 μg/kg, or from about 600 μg/kg to about 800 μg/kg, or from about 400 μg/kg to about 600 μg/kg, or from about 400 μg/kg to about 36,000 μg/kg, or from about 20,000 μg/kg to about 36,000 μg/kg or from about 5000 μg/kg to about 8000 μg/kg, or from about 8000 μg/kg to about 12,000 μg/kg, or from about 10,000 μg/kg to about 20,000 μg/kg, or from about 15,000 μg/kg to about 20,000 μg/kg.

In some embodiments, the grape-derived product contains at least about 5000 μg/kg glutathione-3SH. For example, in some embodiments, the grape-derived product contains at least about 5000 μg/kg, at least about 5500 μg/kg, at least about 6000 μg/kg, at least about 6500 μg/kg, at least about 7000 μg/kg, at least about 7500 μg/kg, at least about 8000 μg/kg, at least about 8500 μg/kg, at least about 9000 μg/kg, at least about 9500 μg/kg, at least about 10,000 μg/kg, at least about 10,500 μg/kg, at least about 11,000 μg/kg, at least about 11,500 μg/kg, at least about 12,000 μg/kg, at least about 12,500 μg/kg, at least about 13,000 μg/kg, at least about 13,500 μg/kg, at least about 14,000 μg/kg, at least 14,500 μg/kg, at last about 15,000 μg/kg, at least about 15,500 μg/kg, at least about 16,000 μg/kg, at least about 16,500 μg/kg, at least about 17,000 μg/kg, at least about 17,500 μg/kg, at least about 18,000 μg/kg, at least about 18,500 μg/kg, at least about 19,000 μg/kg, at least about 19,500 μg/kg, or at least about 20,000 μg/kg, or at least 30,000 μg/kg, or at least 35,00 μg/kg, or at least 40,000 μg/kg, or at least 45,000 μg/kg, or at least 50,000 μg/kg glutathione-bound-3SH. In some embodiments, the grape-derived product contains an amount of glutathione-bound-3SH ranging from about 5000 μg/kg to about 1000 μg/kg, or from about 5500 μg/kg to about 9000 μg/kg, or from about 6000 μg/kg to about 8000 μg/kg, or from about 4000 μg/kg to about 6000 μg/kg, or from about 400 μg/kg to about 36,000 μg/kg, or from about 20,000 μg/kg to about 50,000 μg/kg or from about 5000 μg/kg to about 8000 μg/kg, or from about 8000 μg/kg to about 12,000 μg/kg, or from about 10,000 μg/kg to about 20,000 μg/kg, or from about 15,000 μg/kg to about 20,000 μg/kg.

In some embodiments, the mash hopping step comprises adding less than 100 grams of the plant material per kg of grist (e.g., adding less than 75 grams of the plant material per kg of grist or less than 50 grams of the plant material per kg of grist). In some embodiments, the mash hopping step comprises adding between 75-100 grams of the plant material per 1 kg of grist. In some embodiments, the mash hopping step comprises adding between 50-75 grams of the plant material per 1 kg of grist. In some embodiments, the mash hopping step comprises adding between 25-50 grams of plant material per 1 kg of grist. In some embodiments, the mash hopping step comprises adding between 1-25 grams of plant material per 1 kg of grist. In some embodiments, the mash hopping step comprises adding both hops and a grape-derived product to the grist.

Optionally, the mash hopping step comprises a protein rest before the boiling step. For example, in some embodiments, the protein rest comprises maintaining the mash at a temperature of less than 120° F. (e.g., between 100° F. and 120° F.) for at least five minutes (e.g., at least about 5 minutes, or at least about 10 minutes, or at least about 15 minutes, or at least about 20 minutes, or at least about 25 minutes, or at least about 30 minutes, or at least about 5 minutes, or at least about 40 minutes, or at least about 45 minutes, or at least about 50 minutes, or about one hour before the boiling step. In some embodiments, the protein rest comprises maintaining the mash at a temperature of less than 120° F. (e.g., between 100° F. and 120° F.) for no more than one hour before the boiling step. In some embodiments, the protein rest comprises maintaining the mash at a temperature of less than 120° F. (e.g., between 100° F. and 120° F.) for a time ranging from 5 minutes to one hour (or from about 5 minutes to about 20 minutes, or about 5 minutes to about 10 minutes, or from about 10 minutes to about 30 minutes, or about 10 minutes to about one hour) before the boiling step.

In some embodiments, the mash hopping step further comprises a saccharification rest after the protein rest and before the boiling step. For example, in some embodiments, the saccharification rest comprises maintaining the mash at a temperature of less than 160° F. (e.g., between 140° F. and 160° F.) for at least 15 minutes hour before the boiling step. In some embodiments, the saccharification rest comprises maintaining the mash at a temperature of less than 160° F. (e.g., between 140° F. and 160° F., or between 148° F. and 158° F.) for at least about 15 minutes, or at least about 20 minutes, or at least about 25 minutes, or at least about 30 minutes, or at least about 5 minutes, or at least about 40 minutes, or at least about 45 minutes, or at least about 50 minutes, or at least about 60 minutes, or about 65 minutes, or about 70 minutes, or about 75 minutes, or about 80 minutes, or about 85 minutes or about 90 minutes. In some embodiments, the saccharification rest comprises maintaining the mash at a temperature of less than 160° F. (e.g., between 140° F. and 160° F., or between 148° F. and 158° F.) for a time ranging from 15 minutes to 90 minutes (or from about 15 minutes to about 30 minutes, or about 20 minutes to about 60 minutes, or from about 20 minutes to about 40 minutes, or about 60 minutes to about 90 minutes) before the boiling step.

In various embodiments, the method further comprises contacting the cooled wort with hops to produce an admixture, and contacting the admixture with the recombinant Saccharomyces. In some embodiments, the recombinant Saccharomyces is S. cerevisiae or S. pastorianus.

In some embodiments, the contacting step occurs in a fermenter at a temperature ranging from 45° F.-100° F. In some embodiments, the contacting step occurs for a period of time ranging from 3 days to 14 days (e.g., about 3 days, about 4 days, about 5 days, about 6 days, about 7 days, about 8 days, about 9 days, about 10 days, about 11 days, about 12 days, about 13 days, or about 14 days.

Methods of converting other non-volatile thiols (e.g., glutathione-bound- or cysteine-bound-, non-3SH thiols) in a brewing process are also contemplated. For example, it is contemplated that the methods described herein are useful for converting glutathione bound- or cysteine bound-4MSP, glutathione bound- or cysteine bound-3SH or glutathione bound- or cysteine bound-3SHA) to their aromatic, free forms.

In any of the methods described herein, the wort optionally comprises at least 60 ng/L free 3SH after the contacting step. In some embodiments, the wort comprises about 60 ng/L (or about 65 ng/L, or about 70 ng/L, or about 75 ng/L, or about 80 ng/L, or about 85 ng/L, or about 90 ng/L, or about 95 ng/L, or about 100 ng/L, or about 110 ng/L, or about 120 ng/L, or about 130 ng/L, or about 140 ng/L, or about 150 ng/L, or about 160 ng/L, or about 170 ng/L, or about 180 ng/L, or about 190 ng/L, or about 200 ng/L, or about 250 ng/L, or about 300 ng/L, or about 350 ng/L, or about 400 ng/L, or about 450 ng/L, or about 500 ng/L, or about 550 ng/L, or about 600 ng/L, or about 650 ng/L, or about 700 ng/L, or about 750 ng/L, or about 800 ng/L, or about 850 ng/L, or about 900 ng/L, or about 950 ng/L, or about 1000 ng/L) free 3SH after the contacting step. Free thiol in the sample can be analyzed, for example, by stable isotope dilution assay and nano-liquid chromatography tandem mass spectrometry (Nano LC-MS/MS) (Roland et al., J. Chromatography A, 1468:154-163, 2016, the disclosure of which is incorporated herein by reference). Methods of quantifying an amount of free thiols in a sample can be performed, for example, by derivatization and high-performance liquid chromatography-tandem mass spectrometry (HPLC-MS/MS) (Capone et al., Anal. Chem., 87:1226-1231, 2015, the disclosure of which is incorporated by reference).

In some embodiments, the method results in a 6-fold increase in free 3SH in wort fermented with the recombinant Saccharomyces compared to wort fermented with non-modified Saccharomyces.

### EXAMPLES

### Example 1—Recombinant Saccharomyces

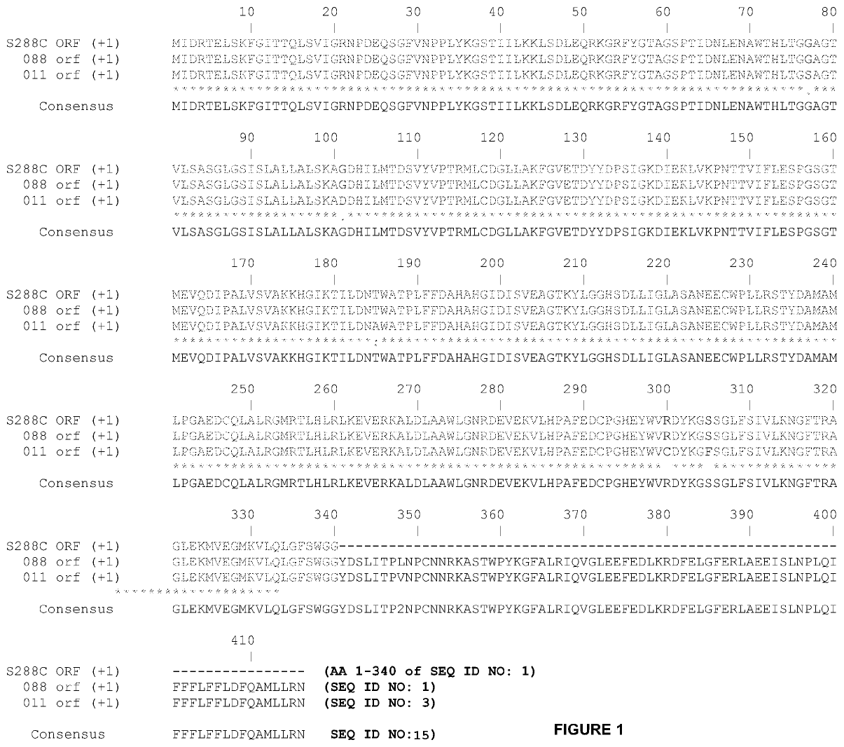

An integration cassette containing the KANMX4 gene along with the TDH3 promoter element was amplified with primers containing 80 bp homology to the IRC7 promoter (SEQ ID NO: 5: 5′-AAAAGGCTCCTGATGAAACTGGAGAGTCTCTTTGTTCTGAAATTTTTAAAGTTTAGCACACCATATAG-3′ (forward primer) and SEQ ID NO: 6: 5′-TCCAATAACAGACAGTTGCGTAGTAATACCAAACTTCGATAACTCGGTACGATCAATCATTTTGTTTGTTTATGTGTGTTTATTCGAAAC-3′ (reverse primer). This cassette was transformed into the respective S. cerevisiae parent strains OYL-088 (WGS of yeast strain A.US-05 (SRR8173067)) and OYL-011 (WGS of yeast strain YMD1872 (SRR8172941)) resulting in a targeted integration of the KANMX-TDH3pr sequence upstream IRC7. PCR confirmation of the resulting G418-resistant colonies verified the successful integration resulting in TDH3 promoter-driven overexpression of IRC7. The expressed IRC7 alleles were further sequenced and aligned to wildtype IRC7 sequence (FIG. 1). SNPs and resulting amino acid substitutions are noted below in Table 1.

TABLE 1

| Amino acid substitutions | | |
|---|---|---|
| from SNPs | OYL-088 | OYL-011 |
| G77S | No | Yes |
| G101D | No | Yes |
| T185A | No | Yes |
| R300C | No | Yes |
| S305F | No | Yes |
| V348L | Yes | No |

Results indicated that the modified OYL-088 strain expressed an active allele of IRC7 (amino acid sequence set forth in SEQ ID NO: 1, polynucleotide sequence set forth in SEQ ID NO: 2), whereas the SNPs present in the polynucleotide encoding IRC7 in the modified OYL-011 strain led to expression of an inactive allele of IRC7 (amino acid sequence set forth in SEQ ID NO: 3, polynucleotide sequence set forth in SEQ ID NO: 4).

### Example 2—Brewing Methods

Using a small pilot brewing system, wort was prepared with a “brew in the bag” or BLAB method. For the mash hopped samples, 2-Row Brewer's Malt (Briess Malting) and Cascade hops (Hopsteiner) were steeped at 120° F. for 15 minutes for a protein rest and then heated to 148° F. for 15 minutes for a saccharification rest. The grain and hops were removed from the wort, and the volume was adjusted to target 10° P at the beginning of the boil. The wort was boiled for 30 minutes and then transferred into a whirlpool stand for an additional 15 before cooling into flasks. The resulting wort (12° P) was fermented with either OYL-088, OYL-011, OYL-088 TDH3-IRC7, or OYL-011 TDH3-IRC7. The same process was performed for the dry hopped samples, but hops were omitted from the mash and later added on the second day of fermentation at the same hopping rate of 8 g/L (˜2 lb/bbl). Wort samples and beer samples were collected in 50 ml conical tubes, 1.5 mg/L of sodium metabisulfite was added to the samples and samples were immediately frozen. The samples were assessed by derivatization and HPLC-MS/MS as described in Capone et al. (Anal. Chem., 87:1226-1231, 2015) and also by stable isotope dilution assay and Nano LC-MS/MS as described by Roland et al. (J. Chromatography A, 1468:154-163, 2016).

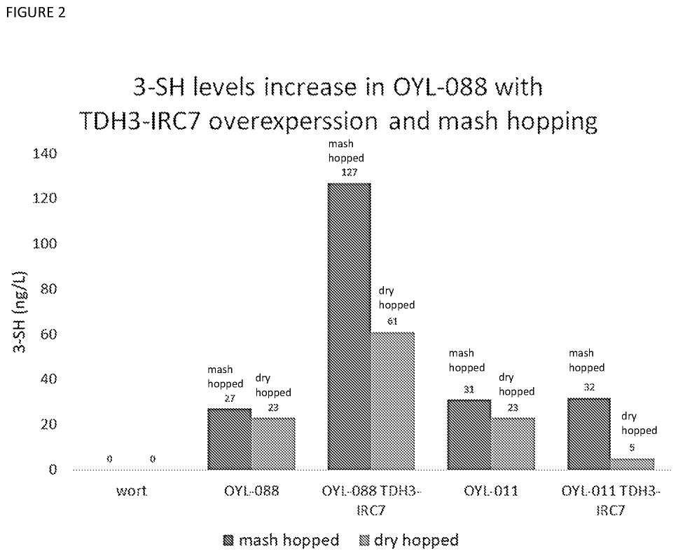

As shown in FIG. 2, in beer samples fermented with OYL-088 TDH3-IRC7, free 3SH levels increased nearly 3-fold compared to the non-modified strain (OYL-088). 3SH levels in the same beer made with OYL-011 and OYL-011 TDH3-IRC7 remained unchanged in comparison (due to its inactive allele of IRC7). Surprisingly, in the mash hopped beer samples fermented with OYL-088 TDH3-IRC7, free 3SH levels increased nearly 6-fold compared to the non-modified strain (OYL-088). 3SH levels in the same beer made with OYL-011 and OYL-011 TDH3-IRC7 remained unchanged in comparison (due to its inactive allele of IRC7).

The results described above were surprising, at least in part, in view of reports that β-lyase overexpression (specifically, IRC7 overexpression) in Saccharomyces does not produce free 3-SH (Denby et al., WBC Connect 2020, Poster 159). In the course of the experiments described herein, it was determined that previous studies utilized an inactive form of IRC7. In another study examining cell extracts for endogenous β-lyase activity (i.e., not an overexpression study), it was determined that Irc7 with the V348L substitution (same as in SEQ ID NO: 1) was inactive (Curtin et al., “Mutations in carbon-sulfur β-lyase encoding gene IRC7 affect the polyfunctional thiol-releasing capability of brewers yeast,” World Brewing Congress Connect 2020, Poster 157). Unexpectedly, and as shown herein, Irc7 comprising an amino acid sequence set forth in SEQ ID NO: 1 does efficiently convert non-volatile thiols available in plant matter into their free, aromatic forms. Additionally, use of active β-lyase in a mash hopping step results in a significant increase in the conversion of non-volatile thiols to their free forms.

### Example 3—Recombinant Yeast

Gblocks were synthesized by IDT and cloned into a yeast shuttle vector with a HYGB selective marker. These Gblocks contained a polynucleotide encoding a PatB from S. hominis (SEQ ID NO. 8), IRC7 from S. cerevisiae (SEQ ID NO. 1), or IRC7 from S. pastorianus (SEQ ID NO. 7) under regulation by the PGK1 promoter and CYC1 terminator. The vectors were then transformed into a lager brewing strain (OYL-106) and isolates were grown on YPD-HYGB agar plates. These isolates were inoculated into YPD+HYGB to assay for sulfur production with lead acetate strips. The lead acetate strips showed significant darkening with the S. pastorianus IRC7 allele, less with the S. cerevisiae IRC7 allele and little to no darkening with the S. hominis PatB allele and empty vector control. The isolates were propagated in 250 ml of dried malt extract medium+HYGB for small flask fermentations. After 48 hours of growth, each propagation culture was centrifuged and 10 million cells/ml were used to inoculate 300 ml of 15° P Wort prepared from 2-row barley malt. The fermentation proceeded for 1 week at which time sensory was performed. The resulting sensory impact of the S. hominis PatB allele was significantly more pronounced than the cerevisiae IRC7 allele and empty vector control. The major aromatic descriptor for the S. hominis PatB fermentation was intense passionfruit, guava and grapefruit, all characteristic of the 3SH thiol.

Investigation into additional PatB alleles was performed in a similar manner described above. Plasmids with PatB alleles from S. anginosus, S. cohnii and B. subtilis (SEQ ID NO: 14) were transformed into the lager brewing strain (OYL-106) and compared to the S. hominis PatB allele. The resulting wort fermentations were assessed by sensory. The fermentation with overexpression of the B. subtilis PatB allele resulted in a comparable level of passionfruit and guava aromas as the S. hominis PatB allele, whereas the fermentations with overexpression of S. anginosus and S. cohnii PatB alleles showed little to no considerable enhancement of aroma relative to the control fermentation with no overexpression.

### Example 4—Comparison of the Effect of PatB Cysteine-Thiol Lyase and Irc7, and TnaA β-Lyase Enzymes on Thiol Release

Trial fermentations were performed to evaluate the activity and specificity of the Irc7, PatB and TnaA β-lyase enzymes. Wort was prepared in a commercial brewhouse by mashing malted 2-row barley at 148° F. for 30 minutes for saccharification followed by a 5 minute inactivation of enzyme activity at 180° F. The runoff of the mash was collected and boiled for 30 minutes. The malt extract was diluted to 15 ° P and cooled to 70° F. prior to inoculating with one of the following: OYL-011, OYL-011+Irc7, OYL-011+PatB or OYL-011+TnaA. Fermentation flasks were configured with a hydrogen sulphide detector tube (4H Gastec) to quantify the cumulative H₂S released throughout fermentation. When fermentation was completed (after 11 days), H₂S levels were recorded, and the resulting beer was analyzed for free thiols 3SH and 4MSP. The results are show below in Table 2.

TABLE 2

| | H₂S | 3SH | 4MSP |
|---|---|---|---|
| | (cumulative ppm) | (ppt) | (ppt) |
| Parent OYL-011 | n.d. | 86 | n.d. |
| OYL-011 + Irc7 | 175 | 696 | n.d. |
| OYL-011 + PatB | n.d. | 10559 | 8.2 |
| OYL-011 + TnaA | n.d. | 10078 | 3.5 |
| Sensory Threshold | 4 | 60 | 1.5 |

n.d. = not determined

As shown above, strains expressing the S. hominis PatB cysteine-thiol lyase have enhanced 3SH output and reduced H₂S output relative to strains expressing the S. cerevisiae Irc7 β-lyase. The S. hominis PatB allele also shows enhanced 4MSP relative to another enzyme with known β-lyase activity, TnaA tryptophanase from C. amalonaticus.

What is claimed is:

1. A recombinant Saccharomyces spp comprising a polynucleotide encoding a yeast β-lyase enzyme Irc7 operably linked to a heterologous promoter, wherein the β-lyase enzyme comprises an amino acid sequence at least 95% identical to the amino acid sequence set forth in SEQ ID NO: 1.

2. The recombinant Saccharomyces spp of claim 1, wherein the heterologous promoter is TDH3, TDH2, CCW12, PGK1, ADH1, ADH2, CYC1, HHF1, HHF2, TEF1, TEF2, HTB2, PAB1, ALD6, RNR1, RNR2, POP6, RAD27, PSP2, REV1, MFA1, MFa2, GAL1, CUP1, MET25, ICL1, ICL2, GAL3, HXT1, HXT2, MAL11, MAL31, MAL32, MAL33, MRK1, or SUC2 promoter.

3. The recombinant Saccharomyces spp of claim 1 or claim 2, that is S. cerevisiae.

4. The recombinant Saccharomyces spp of claim 1 or claim 2, that is S. pastorianus.

5. The recombinant Saccharomyces spp of any one of claims 1-4, wherein the β-lyase enzyme comprises the amino acid sequence set forth in SEQ ID NO: 1.

6. A method of converting a non-volatile form of 3-sulfanyl-1-hexanol (3SH) to free 3SH during a brewing process, the method comprising contacting cooled wort with the recombinant Saccharomyces according to any one of claims 1-5 under conditions and for a time sufficient to convert non-volatile form of 3SH to free 3SH.

7. The method of claim 6, wherein the non-volatile form of 3SH is glutathione-bound-3SH, cysteine-bound-3SH, or a combination thereof

8. The method of claim 6 or claim 7, wherein the method results in a 3-fold increase in free 3SH in wort fermented with the recombinant Saccharomyces compared to wort fermented with unmodified Saccharomyces.

9. The method of any one of claims 6-8, wherein the wort comprises at least 60 ng/L free 3SH after the contacting step.

10. The method of any one of claims 6-9, wherein the method comprises adding hops to cooled wort during the contacting step.

11. The method of claim 10, wherein the hops contain at least 400 μg/kg of cysteine-bound 3SH.

12. The method of claim 10 or claim 11, wherein the hops are Cascade, Calypso, Hallertau Tradition, Hallertau Perle, Triple Pearl, Nugget, Saaz, Columbus/CTZ, Chinook, Nelson Sauvin, Hallertau Blanc or Simcoe hops.

13. A method of converting of a non-volatile form of 3-sulfanyl-1-hexanol (3SH) to free 3SH during a brewing process, the method comprising
(a) a mash hopping step comprising adding a plant comprising a non-volatile form of 3SH to mash to produce wort;
(b) boiling the wort produced by (a);
(c) cooling the wort; and
(d) contacting the cooled wort with a recombinant Saccharomyces for a time sufficient to convert a non-volatile form of 3SH to free 3SH, wherein the recombinant Saccharomyces comprises:
(i) a polynucleotide encoding an active β-lyase enzyme Irc7 operably linked to a heterologous promoter; or
(ii) a polynucleotide encoding a cysteine-thiol lyase operably linked to a heterologous promoter.

14. The method of claim 13, wherein the non-volatile form of 3SH is glutathione-bound-3SH, cysteine bound-3SH, and combinations thereof.

15. The method of claim 13 or claim 14, wherein the plant material comprising a non-volatile form of 3SH is hops.

16. The method of claim 15, wherein the hops contain at least 400 μg/kg of cysteine-bound 3SH.

17. The method of claims 15 or claim 16, wherein the hops contain at least 5000 μg/kg glutathione-3SH.

18. The method of any one of claims 15-17, wherein the hops are Cascade, Calypso, Hallertau Tradition, Hallertau Perle, Triple Pearl, Nugget, Saaz, Columbus/CTZ, Chinook, Nelson Sauvin, Hallertau Blanc or Simcoe hops.

19. The method of claim 13 or claim 14, wherein the plant material comprising a non-volatile form of 3SH is a grape-derived product.

20. The method of claim 19, wherein the grape-derived product is crushed grapes or grape flour.

21. The method of claim 20, wherein the grape-derived product is obtained from a white grape, a red grape, or combination thereof.

22. The method of claim 21, wherein the white grape is Sauvignon Blanc, Chardonnay, Chenin Blanc, Colombard, Gewurztraminer, Gros Manseng, Koshu, Maccabeo, Muscat, Petit Manseng, Pinot Blanc, Pinot Gris, Riesling, Scheurebe, Semillon, Sylvaner, or Tokay.

23. The method of claim 21, wherein the red grape is Cabernet Franc, Cabernet Sauvignon, Grenache, Merlot, or Pinot Noir.

24. The method of any one of claims 13-23, wherein the β-lyase is a bacterial β-lyase or a fungal β-lyase.

25. The method of claim 24, wherein the β-lyase is a bacterial β-lyase.

26. The method of claim 25, wherein the β-lyase is from Eschericia sp.; Thermoanaerobacter sp.; Symbiobacterium sp.; Photobacterium sp.; Haemophilus sp.; Vibrio sp.; Proteus sp.; Halobacterium sp.; Desulfitobacterium sp.; or Treponema sp.

27. The method of claim 25, wherein the bacterial β-lyase is E. coli TNaA.

28. The method of claim 24, wherein the fungal β-lyase is a yeast β-lyase.

29. The method of claim 28, wherein the yeast β-lyase is from Saccharomycotina, Taphrinomycotina, or Schizosaccharomycetes.

30. The method of claim 28, wherein the yeast β-lyase is from Saccharomyces.

31. The method of claim 28, wherein yeast β-lyase is Irc7.

32. The method of claim 31, wherein the Irc7 comprises an amino acid sequence at least 95% identical to the amino acid sequence set forth in SEQ ID NO: 1.

33. The method of claim 31 or claim 32, wherein the Irc7 comprises the amino acid sequence set forth in SEQ ID NO: 1.

34. The method of claims 13-23, wherein the cysteine-thiol lyase is PatB.

35. The method of claim 34, wherein the PatB is from S. lugdunensis, S. devriesei, S. hominis, S. haemolyticus, S. petrasii or B. subtilis.

36. The method of claim 34 or claim 35, wherein the PatB comprises a nucleotide sequence at least 80% identical to the nucleotide sequence set forth in any one of SEQ ID NOs: 8-14.

37. The method of any one of claims 15-36, wherein the method comprises adding less than 100 grams of hops per 1 kg of grist during the mashing step.

38. The method of any one of claim 13-37, wherein mash hopping step comprises a protein rest.

39. The method of claim 38, wherein the protein rest comprises maintaining the mash at a temperature of less than 130° F. for at least 5 minutes before the boiling step.

40. The method of claim 38 or claim 39, wherein the protein rest comprises maintaining the mash at a temperature between 100° and 120° F. for at least 5 minutes before the boiling step.

41. The method of claim 38 or 39, further comprising a saccharification rest after the protein rest and before the boiling step.

42. The method of claim 41, wherein the saccharification rest comprises maintaining the wort at a temperature between 120° and 160° F. for at least 15 minutes before the boiling step.

43. The method of any one of claims 13-42, further comprising contacting the cooled wort with hops to produce an admixture and contacting the admixture with the recombinant Saccharomyces.

44. The method of any one of claims 13-43, wherein the recombinant Saccharomyces is S. cerevisiae or S. pastorianus.

45. The method of any one of claims 13-44, wherein the wort comprises at least 60 ng/L free 3SH after the contacting step.

46. The method of any one of claims 13-45, wherein the contacting step (d) occurs in a fermenter at a temperature ranging from 45° F.-100° F.

47. The method of any one of claims 13-46, wherein the method results in a 6-fold increase in free 3SH in wort fermented with the recombinant S. cerevisiae compared to wort fermented with non-modified S. cerevisiae.

48. A recombinant yeast comprising a polynucleotide encoding a yeast β-lyase enzyme Irc7 operably linked to a heterologous promoter, wherein the β-lyase enzyme comprises an amino acid sequence at least 95% identical to the amino acid sequence set forth in SEQ ID NO: 1.

49. The recombinant yeast of claim 48, wherein the yeast belongs to a non-Saccharomyces genus.

50. The recombinant yeast of claim 48 or clam 49, wherein the yeast is of the genus Kloeckera, Candida, Starmerella, Hanseniaspora, Kluyveromyces/Lachance, Metschnikowia, Saccharomycodes, Zygosaccharomyce, Dekkera (also referred to as Brettanomyces), Wickerhamomyces, or Torulaspora.

50. The recombinant yeast of any one of claims 48-50, wherein the yeast is Hanseniaspora uvarum, Hanseniaspora guillermondii, Hanseniaspora vinae, Metschnikowia pulcherrima, Kluyveromyces/Lachancea thermotolerans, Starmerella bacillaris (previously referred to as Candida stellatal or Candida zemplinina), Saccharomycodes ludwigii, Zygosaccharomyces rouxii, Dekkera bruxellensis, Dekkera anomala, Brettanomyces custersianus, Brettanomyces naardenensis, Brettanomyces nanus, Wickerhamomyces anomalus, or Torulaspora delbrueckii.

51. A recombinant yeast comprising a polynucleotide encoding a cysteine-thiol lyase operably linked to a heterologous promoter.

52. The recombinant yeast of claim 51, wherein the heterologous promoter is TDH3, TDH2, CCW12, PGK1, ADH1, ADH2, CYC1, HHF1, HHF2, TEF1, TEF2, HTB2, PAB1, ALD6, RNR1, RNR2, POP6, RAD27, PSP2, REV1, MFA1, MFa2, GAL1, CUP1, MET25, ICL1, ICL2, GAL3, HXT1, HXT2, MAL11, MAL31, MAL32, MAL33, MRK1, or SUC2 promoter.

53. The recombinant yeast of claim 51 or claim 52, wherein the yeast belong to the Saccharomyces genus.

54. The recombinant yeast of any one of claims 51-53, wherein the yeast is S. cerevisiae or S. pastorianus.

55. The recombinant yeast of claim 51 or claim 52, wherein the yeast belongs to a non-Saccharomyces genus.

56. The recombinant yeast of claim 55, wherein the yeast is of the genus Kloeckera, Candida, Starmerella, Hanseniaspora, Kluyveromyces/Lachance, Metschnikowia, Saccharomycodes, Zygosaccharomyce, Dekkera (also referred to as Brettanomyces), Wickerhamomyces, or Torulaspora.

57. The recombinant yeast of claims 55 or claim 56, wherein the yeast is Hanseniaspora uvarum, Hanseniaspora guillermondii, Hanseniaspora vinae, Metschnikowia pulcherrima, Kluyveromyces/Lachancea thermotolerans, Starmerella bacillaris (previously referred to as Candida stellatal or Candida zemplinina), Saccharomycodes ludwigii, Zygosaccharomyces rouxii, Dekkera bruxellensis, Dekkera anomala, Brettanomyces custersianus, Brettanomyces naardenensis, Brettanomyces nanus, Wickerhamomyces anomalus, or Torulaspora delbrueckii.

58. The recombinant yeast of any one of claims 51-57, wherein the cysteine-thiol lyase is PatB.

59. The recombinant yeast of claim 58, wherein the PatB is from S. lugdunensis, S. devriesei, S. hominis, S. haemolyticus, S. petrasii or B. subtilis.

60. The recombinant yeast of claim 58 or claim 59, wherein the PatB comprises a nucleotide sequence at least 80% identical to the nucleotide sequence set forth in any one of SEQ ID NOs: 8-14.

61. A method of converting a non-volatile form of 3-sulfanyl-1-hexanol (3SH) to free 3SH during a brewing process, the method comprising contacting a fermentable sugar source with the recombinant yeast according to any one of claims 51-60 under conditions and for a time sufficient to convert non-volatile form of 3SH to free 3SH.

62. The method of claim 61, wherein the non-volatile form of 3SH is glutathione-bound-3SH, cysteine-bound-3SH, or a combination thereof.

63. The method of claim 61 or claim 62, wherein the method results in a 3-fold increase in free 3SH in a fermentable sugar source fermented with the recombinant yeast compared to fermentable sugar fermented with unmodified yeast.

64. The method of claim 64, wherein the fermentable sugar source is wort.

65. The method of any one of claims 61-63, wherein the wort comprises at least 60 ng/L free 3SH after the contacting step.

66. The method of claim 64 or claim 65, wherein the method comprises adding hops to cooled wort during the contacting step.

67. The method of claim 66, wherein the hops contain at least 400 μg/kg of cysteine-bound 3SH.

68. The method of claim 66 or claim 67, wherein the hops are Cascade, Calypso, Hallertau Tradition, Hallertau Perle, Triple Pearl, Nugget, Saaz, Columbus/CTZ, Chinook, Nelson Sauvin, Hallertau Blanc or Simcoe hops.
